(19) United States
(12) Patent Application Publication
(10) Pub. No.: US 2024/0364859 A1
(43) Pub. Date: Oct. 31, 2024

(54) METHOD FOR STEREO IMAGE PROCESSING AND REPRESENTATION AND SYSTEM

(71) Applicant: Siemens Healthineers AG, Forchheim (DE)

(72) Inventor: Alois Regensburger, Poxdorf (DE)

(21) Appl. No.: 18/645,593

(22) Filed: Apr. 25, 2024

(30) Foreign Application Priority Data
Apr. 28, 2023 (DE) 10 2023 203 957.5

(51) Int. Cl. H04N 13/398; G06T 3/40; G06T 5/20; G06T 5/70; G06T 5/73; G06T 7/50; H04N 13/383

(52) U.S. Cl. CPC H04N 13/398; G06T 3/40; G06T 5/20; G06T 5/70; G06T 5/73; G06T 7/50; H04N 13/383

(57) ABSTRACT

For an improved stereopsis, a method and system is provided for stereo image processing and representation of one stereo image for a predetermined person. The stereo image includes (at least) one first partial image and one second partial image, which partial images represent different projection directions and are associated respectively with a right eye and a left eye of the person. The method includes: providing visual capacity data of the right eye and visual capacity data of the left eye and/or visual capacity data of both eyes of the predetermined person, processing the stereo image such that at least one corresponding image feature in the first partial image and in the second partial image is amended in the first partial image and the second partial image, respectively, dependent upon the visual capacity data of the respective associated eye of the predetermined person, and displaying the stereo image, processed in this manner, by a stereoscopic display apparatus.

### CROSS REFERENCE TO RELATED APPLICATIONS

This application claims the benefit of DE 10 2023 203 957.5 filed on Apr. 28, 2023, which is hereby incorporated by reference in its entirety.

### FIELD

Embodiments relate to a method for stereo image processing and representation of a stereo image for a predetermined person.

### BACKGROUND

A large number of people (approximately a third of the population) have difficulties, when viewing stereo representations, in recognizing a depth impression (stereopsis). Only a fraction of these people are unable to process stereo information at all, while most of those affected have merely a reduced capability for perceiving and processing stereoscopic depth impressions. In particular, if the visual capacity of a person is different between the two eyes, difficulties arise in the brain in reliably carrying out the stereo correspondence analysis that is necessary for a stereo depth impression. For example, despite a visual acuity correction with aids (spectacles, contact lenses), one of the two eyes may have a reduced spatial resolution or contrast resolution. In everyday life, the people affected typically do not notice their reduced capacity to observe scenes in stereo, or barely. Due to a large number of monoscopic depth indications, however, the approximate distances from the eye of the different objects in the space may be attributed to them by the brain.

However, when observing artificial scenes on stereoscopic displays or with VR/AR headsets, for example in a medical environment, this relation to the everyday world often breaks down and the reduced capacity of the brain/visual apparatus to process stereoscopic depth information becomes disadvantageously noticeable. Since the stereoscopic representations come from a situation that is alien to the everyday world of experience such as, for example, from laparoscopy, X-ray, virtual 3D representations or overlays/computations of virtual medical 3D representations with real videos, this reduced capacity however becomes very much more clearly noticeable. The brain is not used to the geometrical information and the other senses such as balance, the accommodation system of the eyes and the possibility of moving the feet or the head in space and thereby to “experience” depth impressions are partially or largely lacking here. Many observers of medical stereo systems (for example endoscopy, laparoscopy, AR/VR headsets) say that they “do not like” the stereo impression or they cannot see well in stereo, and resort to conventional 2D representations. In this way, all the advantages of medical stereoscopic images for diagnosis and treatment are lost.

From Fusion Optics from Leica (explained, for example, here: https://www.youtube.com/watch?v=−4mEO6APca8), it is known in a binocular optical microscope to display to one eye an image with greater depth of focus, but to the other eye an image with a greater resolution in the region of the focal plane. Many observers succeed in “merging” an image in their perception apparatus that has both a large depth of focus and also a high resolution in the region of the plane of focus.

### BRIEF SUMMARY AND DESCRIPTION

The scope of the present disclosure is defined solely by the claims and is not affected to any degree by the statements within this summary. The present embodiments may obviate one or more of the drawbacks or limitations in the related art. Independent of the grammatical term usage, individuals with male, female or other gender identities are included within the term.

Embodiments provide a method that for persons with restricted visual capacity such as a visual disorder in one or both eyes enables a high-quality stereo imaging, including for example, in the medical field.

The method for stereo image processing and representation of (at least) one stereo image for a predetermined person, the stereo image including (at least) one first partial image and one second partial image, which partial images represent different projection directions and are associated respectively with a right eye and a left eye of the person, includes the following steps: providing visual capacity data for the right eye and visual capacity data for the left eye and/or visual capacity data for both eyes of the predetermined person, processing the stereo image such that at least one corresponding image feature in the first partial image and in the second partial image is amended in the first partial image and the second partial image, respectively, dependent upon the visual capacity data of the respective associated eye of the predetermined person, and displaying the stereo image, processed in this manner, by a stereoscopic display apparatus. The stereoscopic image is individually adapted to a particular person and/or their visual capacities, specifically such that each partial image is amended so that it takes account of the visual capacity of the associated eye (the eye that is intended for observing the respective partial image).

The better corresponding features with stereo disparity in the two (for example left and right) partial image(s) may be perceived by a person, the more easily the human perception apparatus is able to recognize and/or to process stereo depth impressions. Weakly textured scenes and/or details that are difficult to perceive make the stereo impression more difficult. In persons with different visual capacity in the two eyes, there is often a “leading eye” that is used in the brain for a majority of the visual perception. The image information for the weaker eye, by contrast, is taken into account less or hardly at all. Information from the weaker eye is therefore processed only for “enhancement” and/or for additional information for the image from the leading eye.

The concept, known in medical imaging, of the projection direction (when recording images, the direction of the central beam starting from the focus point to the mid point of the sensor area of the X-ray detector) should be taken, generalizing to the overall optical system, to denote the central projection axis and/or the angle of view of the corresponding recording. In general, a stereo image is composed of two partial images (a stereo image pair), one for the left eye and one for the right eye, each recorded from slightly different projection directions (for example a few degrees difference). However, stereo images with three or more partial images are also known.

For a person whose stereopsis is restricted due to visual faults, a readily recognizable high quality stereo image may be generated and displayed. For example, an asymmetrical visual fault in the person that occurs only in one eye or particularly severely in one eye and restricts the capability for stereopsis may be compensated for. For such a visually restricted person, a possibility is provided by embodiments, in the medical environment in the case of examinations with stereoscopy, for example laparoscopy, X-ray stereoscopy, virtual 3D representations or overlays/computations of virtual 3D representations with real videos, to be able to provide a well-founded diagnosis and/or to conduct treatment on the basis of all the available information without disadvantages. This is very important, for example, for clinicians or other medical personnel. Persons who have previously dispensed with stereo images due to their restricted visual capacity are thus more willing to make use of this possibility. Tiring of the eyes may also be avoided and/or at least delayed. In this way, the examination and/or treatment workflow is improved and accelerated since additional auxiliary representations are not needed to compensate for the stereo representation. An improved patient care is thus provided in the medical environment.

According to an embodiment, a large number of corresponding image features in the first partial image and the second partial image are amended in the first partial image and in the second partial image, respectively, dependent upon the visual capacity data of the associated eye of the predetermined person. In general, more than one image feature is relevant in order to obtain a good stereo impression. An image feature in this regard is understood to be, for example, an edge, a structure, a line, a corner and/or a whole object.

According to an embodiment, at least one of the following amendments is carried out: enhancing, attenuating, sharpening, blurring, using high pass, using low pass, filtering, increasing or decreasing depth of focus, increasing or decreasing resolution, transforming, sharpening edges, enhancing contrast, adapting brightness, removing small details. Dependent upon the visual capacity of the respective associated eye, one or more of these amendments to the corresponding partial image may bring about a significant improvement in the stereopsis of the person.

The visual capacity data includes a contrast resolution capacity, a stereopsis capacity, a resolving power, an accommodation, a color vision capability, an eye motor function, a visual acuity, a glare sensitivity and/or a low-light vision. The more exactly the visual capacity of the eyes is described, the better a compensation for the restrictions may be carried out using amendments to the respective partial image. In particular, the visual capacity of the eyes of the person may also be established with one or more vision tests.

According to an embodiment, an asymmetrical processing is carried out dependent upon different visual capacities of the two eyes. Generally, in the event that a person has greatly different visual capacities in one eye as compared with the other eye, a severe restriction of the stereoscopic capability is to be expected. If the partial image intended for the significantly worse eye is severely, or at least more severely, processed than the partial image intended for the better eye, then a significant improvement of the stereoscopic capability is to be expected.

According to a further embodiment, a current observation geometry, for example a size of the display apparatus and/or a viewing angle of the person to the display apparatus and/or a spacing of the eyes of the person from the display apparatus is captured and taken into account in the context of the processing of the stereo image. The capture may take place by sensors such as, for example, cameras. The taking into account may be such that the stereo image reflects the actual observation geometry. Thus, for example, the stereo image may be displayed on the display apparatus such that even in a top view, the person sees it at an acute viewing angle as if he were viewing it frontally or that it is displayed, zoomed in, at a large spacing from the display apparatus. The taking into account of the observation geometry may also take place such that the image frequencies and/or structural sizes in the pixel space of the image to be displayed, that may be resolved by the respective eye of the person, are used. Thus, a postprocessing, as described in other embodiments, may be adapted particularly well to the actual perceptibility of the pixels and/or structures displayed on the display unit.

According to a further embodiment, data regarding lighting conditions in the region of the person or the display apparatus and/or optical parameters of the display apparatus, for example the visibility and/or contrast and/or brightness are taken into account during the processing of the stereo image. The capture may take place by sensors such as, for example, cameras or photodiodes. Thus, for example, in dark surroundings, the display may be dimmed or, under front light, turned brighter. For example, the maximum brightness may be adapted to the glare sensitivity of the respective eye, so that dark image regions adjacent to a bright image region are not covered over in the eye/within the eye by dazzling/scattering of the bright image signal. This is advantageous, for example, in the case of lens clouding (cataract) in the left and/or the right eye.

According to a further embodiment, for processing, at least the following steps are carried out: forward convolution of the at least one feature or of the image portion containing the feature of the first partial image and/or of the second partial image with a function describing the visual capacity of the respective associated eye, resulting in a perception representation of the feature by the person, amending the perception representation of the feature through to a desired target perception representation and backwards convolution of the desired target perception representation of the amended feature. By way of the forward convolution, therefore for each partial image individually, firstly the optical impression that a person has on the basis of his restricted visual capacities with the relevant associated eye, is generated artificially, amended to the desired optical impression and then transformed back again by a backwards convolution. The feature may be represented so that the person with his restricted visual capacity may achieve the target perception representation by way of observation. For example, it may be achieved thereby that an improved stereo perception of the image or the scene is enabled for the person, despite his restricted visual capacity, on the basis of the total set of the already perceptible non-modified features and the features made perceptible by way of the digital modification described.

According to a further embodiment, the following steps are carried out for determining the image feature(s) to be processed: analyzing the partial images with regard to corresponding image features and therefrom determining stereo disparities, creating a depth map on the basis of the corresponding features or the whole scene (sum of all features), evaluating the detected corresponding image features with regard to at least one evaluation criterion, and selecting at least one corresponding image feature from the set of detected corresponding image features on the basis of the at least one evaluation criterion. In this way, important image features, for example for the stereo image impression, may be obtained automatically. As compared with a processing of the entire image, time and computing power is thereby saved. The evaluation criteria may be varied as needed, dependent upon the application.

According to a further embodiment, the at least one evaluation criterion has an evaluation regarding the visibility for an average person or the selected person and the corresponding image features which undershoot a threshold value with regard to the visibility criterion are selected. In this way, image features that are able to make only little or hardly any contribution to the stereo impression because, for example, they are not visible anyway for the person or they make no contribution to a fundamental stereo perception may be sorted out. This, in turn, saves time and effort.

According to a further embodiment, a further or alternative evaluation criterion may be applied that has an evaluation of the image features regarding the relevance and the corresponding image features that exceed a threshold value with regard to the visibility criterion are selected. A preselection of important image features may be made so that time and effort is also saved. This may also, for example, be used such that the evaluation criterion is used as a precondition for a corresponding stereo image processing. If particular features that are important for the stereo perception of the stereo image or the scene are not visible or only with difficulty for at least one eye of the person, then they are enhanced or adapted digitally for the relevant eye or for both eyes.

According to an embodiment, a large number and/or a series of stereo images are processed and displayed, that is for example, a video sequence or an image series. In the case of a series of this type, the same image features may be identically treated in successive stereo images in order to simplify and accelerate the method.

Embodiments further provide a system for carrying out the method, the system including a control unit for controlling the method, a data processing unit and an image processing unit for processing the visual capacity data and amending corresponding features in the partial images of a stereo image dependent upon the visual capacity data, a stereoscopic display apparatus for the display of stereo images, and a storage unit for storing at least one stereo image.

According to an embodiment, a test apparatus for testing the visual capacity of the eyes of the person is also associated with the system. In an advantageous manner, the system includes a sensor for measuring the observation geometry and/or a sensor for measuring the lighting conditions.

The system may be configured as a medical system including a medical imaging system for recording and making available a medical stereo image, for example an X-ray device, an ultrasonic device, or an endoscopy device.

Embodiments are described in greater detail on the basis of embodiments illustrated schematically in the drawings, but without any restriction to these embodiments arising therefrom.

### BRIEF DESCRIPTION OF THE FIGURES

FIG. 1 depicts a sequence of steps of a method for stereo image processing and representation according to an embodiment.

FIG. 2 depicts a sequence of substeps of the second step shown in FIG. 1 for processing corresponding features of the partial images of the stereo image according to an embodiment.

FIG. 3 depicts a further sequence of steps of a method for stereo image processing and representation according to an embodiment.

FIG. 4 depicts a sequence of substeps of the fourth step shown in FIG. 3 for selecting features according to an embodiment.

FIG. 5 depicts a sequence of substeps according to FIG. 2 with intermediate results according to an embodiment.

FIG. 6 depicts a view of second partial images of a stereo image for different eyes of a person according to an embodiment.

FIG. 7 depicts a system for carrying out the method according to an embodiment.

### DETAILED DESCRIPTION

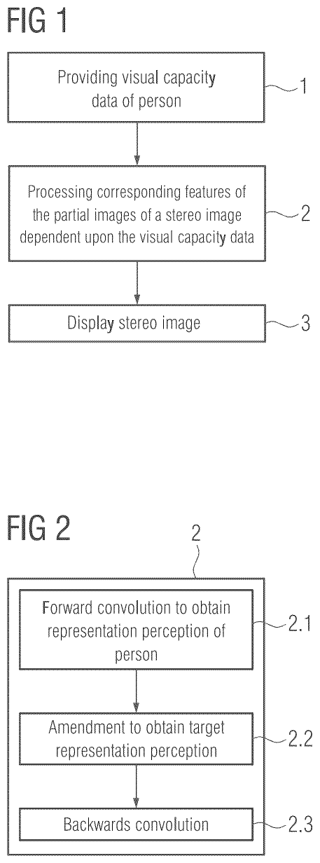

Depicted in FIG. 1 are steps of a method for stereo image processing and representation of one (or more) stereo images for a predetermined person.

A stereo image includes at least one first partial image 30 and one second partial image 31 (for exactly one first and one second partial image: stereo image pair)—see FIG. 6. The at least two partial images represent different projection directions during the image recording, that is for example, a viewing angle difference similar to the average eye. One partial image is associated, for example, with the right eye and the other is associated with the left eye of the person and are provided for viewing and are accordingly displayed on a stereo display apparatus. The respective corresponding image features differ from one another in the partial images according to the projection directions.

While a person with bilaterally fault-free vision receives a three-dimensional impression in his brain when viewing a stereo image, this is often not the case if there is a restriction in the visual capacity of at least one eye. It is particularly difficult if the two eyes differ significantly in their visual capacity; this is the case in many persons despite vision aids (spectacles/contact lenses). Human eyes differ almost always to at least a small degree and often also to a large degree, so that many humans have problems with the viewing of stereo images. By way of the method, a possibility is created for improving the (stereoscopic) depth perception for observers whose visual capacity is weak in at least one eye and/or significantly different in both eyes.

In a first step 1, initially visual capacity data regarding the person who will observe the stereo image is provided. The visual capacity data therein relates to both eyes, that is the left eye and the right eye of the person. As visual capacity data, for example, a contrast resolution capacity, a resolving power, an accommodation, a color vision capability, an eye motor function, a visual acuity and/or a low-light vision may be available and provided. In addition, stereoscopic capability data for both eyes individually or in the interaction of the combined eye pair may also be available and provided. The visual capacity data may be taken from a database or a memory store (for example if the predetermined person is identified, for example, by way of registration in an organ program or suchlike), input directly (for example by the predetermined person himself) or established in advance by one or more vision tests. In the (current or past) establishment of the visual capacity data, a vision aid may be used or it may be established without a vision aid.

In principle, the stereo representation is carried out, in each case, for a particular person whose visual capacity data is then provided. If the visual capacity data of another person, or false visual capacity data, is provided, then the predetermined person probably cannot receive an optimal stereo representation and/or a significantly worse stereo representation might even be expected.

In addition, parameters for a desired enhancement of corresponding image features or stereo depth impressions may also be provided.

In a further step 2, the stereo image is processed, specifically such that at least one corresponding image feature in the first partial image and the second partial image of the stereo image are each amended in the first partial image and the second partial image dependent upon the visual capacity data of the associated eye of the predetermined person. Thus, for example, the corresponding feature in the first partial image that is associated with the first eye is processed, taking account of the visual capacity data of the first eye, and the same feature in the second partial image that is associated with the second eye is processed, taking account of the visual capacity of the second eye. In general, the respective corresponding image feature in the two partial images is processed asymmetrically since the visual capacity data of the two eyes differs in most cases, indeed often significantly. It may be that image features in one of the partial images must be processed only very slightly or not at all if the associated eye has, for example, normal vision (without, or with only slight, restrictions or with the help of a vision aid).

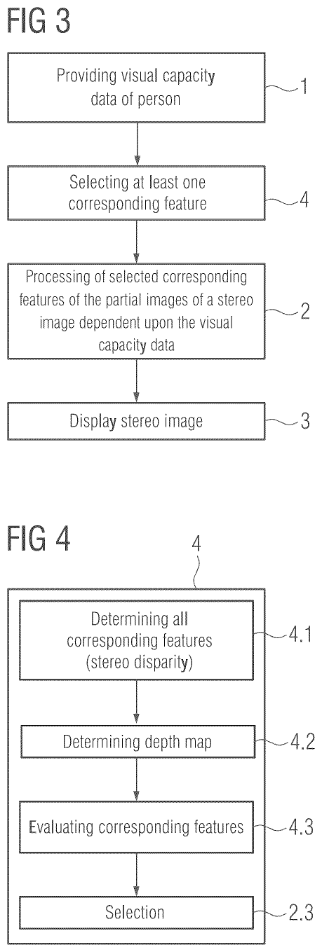

Exactly how many or which corresponding image features are processed may, for example, be determined in advance (see below in the description relating to FIG. 3). In general, more than one image feature is relevant in order to obtain a good stereo impression. An image feature in this context is understood to be, for example, an edge, a structure, a line, a corner, a color change, a color transition and/or an object.

If the processing of the partial images is carried out separately for individual corresponding image features, a combination of a synthetic or modified stereo image from the partial images for the left and right eye with the processed image features is possibly necessary. This is carried out subsequently to the processing.

If the stereo image and/or the at least two partial images are processed accordingly and are possibly combined, in a third step 3, the stereo image is displayed on a display apparatus for stereo images. This may involve, for example, a stereo display, a VR or AR headset, a 3D monitor, or an autostereoscopic display with eye tracking.

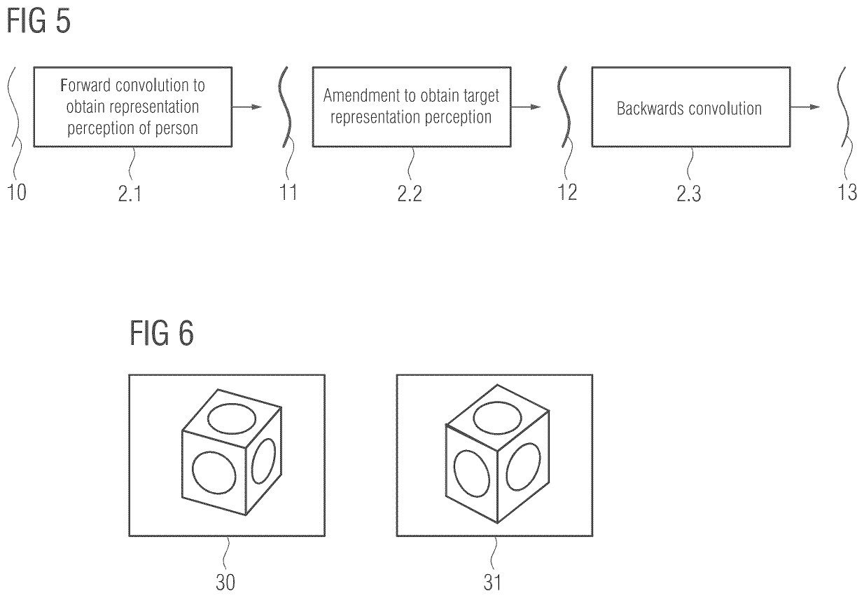

An example of a processing of the stereo images is described in relation to FIGS. 2 and 5. For example, this is described only for a corresponding image feature, but may be carried out, if needed, with as many selected corresponding image features as desired.

In a first substep 2.1, a forward convolution or mapping of the image feature to be processed with a convolution kernel that describes or approximates the visual capacity of the respective eye with the current observation geometry is carried out. This may involve, for example, an optical transfer function, a modulation transfer function (MTF) or a point spread function (PSF). Alternatively, the whole partial image may also be processed with all the image features or a portion of the partial image with a large number of image features. The result of this forward convolution is an estimation of the representation perception 11 of the image feature (or a plurality and/or all image features) by the person with the correspondingly associated eye, that is, a representation of the image feature (or the whole partial image) as the person sees it without any vision aid.

In a second substep 2.2, subsequently a digital amendment/enhancement of the representation perception 11 of the feature (or a plurality and/or all features), for example in the form of a contrast increase is carried out until a target perception 12 (that is the desired representation for the person) of the image feature is obtained. In advance thereof, an evaluation of the representation perception may be carried out, for example on the basis of one or more criteria, as to whether the visibility of the image feature(s) is or are sufficient. If this is the case for one or more image features, then no amendment or enhancement takes place for this.

In a third substep 2.3, the target perception 12 of the image feature (that is, the correspondingly enhanced or amended image feature or portion of the partial image or the whole partial image) is then processed by a backwards convolution or inverse mapping, so that a modified representation 13 of the image feature emerges therein. If, from the modified representations 13 of the at least two partial images, the stereo image is then created and displayed, then the predetermined person may see a particularly good stereo representation despite his restricted visual capacity.

The result of the third substep may be broader than the original image feature. The choice of the inverse convolution kernel or the inverse mapping may take account of the resolution limits of the display apparatus that is to be used and takes account of possible sampling artifacts. Since the inverse convolution is not unambiguously defined, above all, given a poor visual capacity of the person, there exist several degrees of freedom in the configuration of the convolution and the inverse convolution. The operations and results may also only be approximate.

If, particularly large deviations are present in the representation perceptions 11 or the target perceptions 12 of the corresponding image features or the processed partial images between the (for example both the left and the right) partial image, before or after the backwards convolution for the partial image, that are associated with the eye having the better visual capacity (the better-seeing eye), the contrasts of the image feature(s) may be adapted to the partial image associated with the other eye. In order not to influence the overall image perception of the stereo image too much, depending upon the scene/application, it is in general carried out seldom or only to a limited extent.

Apart from the example shown in FIG. 2 for a processing by convolution and backwards convolution, the processing may be carried out in a variety of ways. Thus, according to a further example, the processing may take place by contrast enhancement and possibly broadening the image feature, dependent upon the visual capacity of the person, directly in the original stereo image or the original partial images. This may be carried out, for example, by algorithms for adaptive edge enhancement (edge sharpening).

The stereo images may have been recorded in the medical environment by an imaging device, for example, by an X-ray device with slight tilting of the recording system (for example a C-arm) or by the camera of an endoscope or a laparoscope or by an ultrasonic device. The stereo images may also be generated by the rendering of a virtual 3D scene by way of a data processing unit.

In the context of the method, the observation geometry such as, for example, the size of a display apparatus and/or a viewing angle of the person toward the display apparatus and/or a spacing of the eyes of the person from the display apparatus may also be taken into account during the processing of the partial images, since this may also have an influence on the visual capacity of the person. These values may be captured, for example, by a sensor (spacing sensor, angle sensor, etc.). Alternatively, standard values may also be assumed and used, for example, such that a particular angle element corresponds to a pixel in the partial image/stereo image.

The lighting conditions, for example, data regarding lighting conditions in the region of the person or the display apparatus and/or optical parameters of the display apparatus, for example the visibility and/or contrast and/or brightness, are taken into account during the processing of the partial images. These also have an influence, in general, on visual capacity, for example, they may be restricted in relatively dark surroundings.

If no visual capacity data is available, a vision test may also be carried out to obtain the visual capacity data. Thus, for example, using conventional sight test methods with or without the use of a vision aid (spectacles, contact lenses), the visual capacity and contrast resolving power of both eyes of the predetermined person may be determined. In addition, in order to receive visual capacity data relating to stereoscopic capability, the following tests may be carried out: A plurality of synthetic test scenes or test images are shown in which the two stereo perspectives have differently pronounced corresponding image features. The test scenes or test images may be specifically adapted to the stereo image(s) that are used in the method. Thus, the visual capacity of both eyes determined in advance or input by conventional vision tests may be used as a starting point. The severity of the manipulation of the corresponding image features is selected by the person and this enables, for the person, the individually best compromise between a good image impression and a noticeable depth perception. These parameters (for example, the desired degree of enhancement of corresponding image features) may then be used for the method.

FIGS. 3 and 4 show that, and how, a selection of at least one corresponding feature may be made for a processing. In FIG. 3, in a fourth step 4 that takes place before the second step 2, a selection of at least one, a plurality, or even all corresponding image features is made. In FIG. 4, some substeps of the fourth step 4 are shown. The substeps may be carried out, for example, for a partial image pair of a stereo image recorded with a stereo camera, optionally also for a series (sequence) of stereo images that have been recorded, for example, by a laparoscope or an endoscope.

In a fourth substep 4.1, an analysis of the partial images is carried out with regard to corresponding image features (stereo correspondence analysis or stereo reconstruction) and therefrom a determination of stereo disparities is carried out. This step may be carried out, for example, by known algorithms, such as for example, Libelas (Library for Efficient Large-Scale Stereo Matching) or machine learning methods. In the case of stereo image sequences, the movements of corresponding image features over a plurality of temporal image frames (stereo images in sequence) are taken into account. In a fifth substep 4.2, from the stereo disparities, a so-called depth map of the corresponding features or of the whole scene is generated. A procedure of this type is known.

In a sixth substep 4.3, an evaluation of the detected corresponding image features are carried out with regard to at least one evaluation criterion. The evaluation criterion may be, for example, an evaluation criterion regarding the visibility, that is for example, whether the image features are visible with a particular resolving power and/or contrast resolution and/or color vision capability of the respective eye of a person or what resolving power and/or contrast resolution is necessary in order to see the image features. Herein, the observation geometry and/or the lighting conditions may also be included. The evaluation of the image features may be carried out, for example, for each partial image and the associated eye. A further evaluation criterion may also be a relevance of the image features, that is, the image features may be classified, for example, in two or more steps as to how relevant they are for a depth allocation of the stereo image.

In a seventh substep 4.4, on the basis of at least one evaluation criterion, a selection is then made of at least one corresponding image feature from the set of the detected corresponding image features. Thus, here for example, the corresponding image features may be selected for which the evaluation criterion regarding visibility is barely visible or not at all visible in a partial image, that is, the corresponding image features that, taking account of the visual capability and/or the individual capability for stereo perception in at least one eye, is barely visible and/or detectable and/or differentiable for the person. The set of selected corresponding image features may be a subset of all the corresponding image features or a subset of all the relevant corresponding image features. The correspondingly selected, for example, barely visible corresponding image features are then amended in the second step 2 by targeted digital image processing so that the correspondence analysis is simplified for an observer with his individual visual capacity.

When a sequence of stereo images (scene) is processed, it may optionally be ensured such that a temporal consistency of the appearance of the image features by a plurality of temporal video frames is assured.

At least one step or substep of the method may be carried out by one or more trained machine-learning algorithms.

In another embodiment (synthetic rendering), from one or more stereo images of a scene, first of all, a 3D surface model and/or a textured depth map (RGB+D) of the scene is calculated, for example via neural radiance fields (NeRF), SLAM algorithms or similar known methods. In the calculation of the two partial images for the left and right eye, at positions in the partial images that are relevant for the depth impression at which, for example, only weak, or no, recognizable corresponding image features for the respective eye of the person are present, the image features are enhanced as described above. Alternatively or additionally, at these positions on the partial images, monoscopic image impressions such as color gradients, lighting effects or suchlike may be added in order to enhance the depth impression. Furthermore, virtual overlay contents (for example a colored height grid network) may be added at positions in the stereo image at which the depth perception is difficult for the observer. The overlay contents may also be represented to be readily perceptible by taking account of the visual capacity of the two eyes of the person and the observation geometry as described above.

In a further embodiment, in the representation of virtual 3D objects (for example meshes, volume rendering, etc.) on a stereo output (for example a stereo display), previously corresponding image features are analyzed in the later representation. Subsequently, these image features are selectively enhanced, as described above, adapted to the visual capacity of the eyes, so that the stereo correspondences of the human perception apparatus may be reliably found. Furthermore, as shown above, a suitable virtual illumination of the scene or other methods for the enhancement of monoscopic depth impressions may be selected.

The deviation in stereography denotes the different horizontal spacing of the same image elements in the two partial images from one another and is therefore the mapping of the parallax. Deviation is nowadays increasingly also known as lateral disparity or disparity. Dependent upon the width of the image, deviation is known as relative deviation.

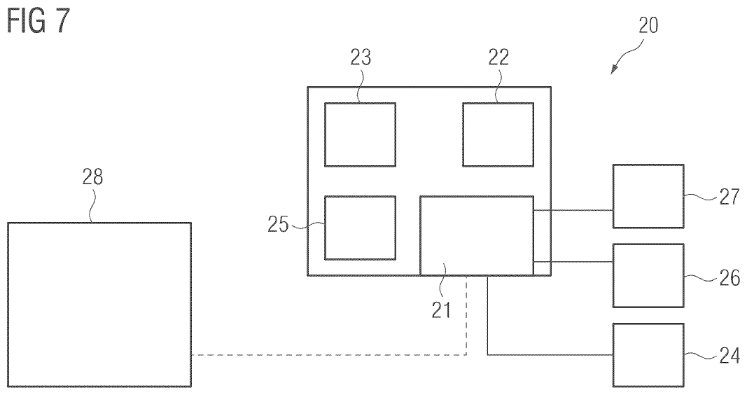

FIG. 7 depicts a system 20 for carrying out the method described. A medical imaging device 28 may be connected thereto that generates medical stereo images and/or partial images, for example, an X-ray device, an endoscope, a laparoscope, or an ultrasound device. The system 20 includes a control unit 21 for controlling the method. The system 20 also includes a data processing unit 22 and an image processing unit 23 for processing the visual capacity data and amending corresponding features in the partial images of the stereo image dependent upon the visual capacity data, as well as a storage unit 25 for storing stereo images and other data. In addition, a stereoscopic display apparatus 24 is provided. This may involve, for example, a stereo display, a VR or AR headset, a 3D monitor, or an autostereoscopic display with eye tracking.

In addition, a test apparatus 27 for testing the visual capacity of the eyes of the person may also be provided. This may be associated with the system 20 or at least configured to send the test result to the system 20. Furthermore, a sensor—for example, in the form of a camera 26—may be provided that measures the observation geometry and/or the lighting conditions.

The method contributes to an expansion of the user group of medical 3D displays and AR/VR headsets. For the observer of the processed stereo images, the depth impression is improved and the effort involved in observing stereo images and scenes is reduced. For the person, an improved 3D perceptibility of “mixed reality” representations of a medical video image and virtual image contents may be achieved.

The embodiments may be briefly summarized as follows: for improved stereopsis, a method for stereo image processing and representation of (at least) one stereo image for a predetermined person, the stereo image including (at least) one first partial image and one second partial image, which partial images represent different projection directions and are associated respectively with a right eye and a left eye of the person, is provided with the following steps: providing visual capacity data of the right eye and visual capacity data of the left eye and/or visual capacity data of both eyes of the predetermined person, processing the stereo image such that at least one corresponding image feature in the first partial image and in the second partial image is amended in the first partial image and the second partial image, respectively, dependent upon the visual capacity data of the respective associated eye of the predetermined person, and displaying the stereo image, processed in this manner, by a stereoscopic display apparatus.

It is to be understood that the elements and features recited in the appended claims may be combined in different ways to produce new claims that likewise fall within the scope of the present disclosure. Thus, whereas the dependent claims appended below depend from only a single independent or dependent claim, it is to be understood that the dependent claims may, alternatively, be made to depend in the alternative from any preceding or following claim, whether independent or dependent, and that such new combinations are to be understood as forming a part of the present specification.

While the present disclosure has been described above by reference to various embodiments, it may be understood that many changes and modifications may be made to the described embodiments. It is therefore intended that the foregoing description be regarded as illustrative rather than limiting, and that it be understood that all equivalents and/or combinations of embodiments are intended to be included in this description.

1. A method for stereo image processing and representation of at least one stereo image for a predetermined person, the at least one stereo image comprising one first partial image and one second partial image, wherein the one first partial image and one second partial image represent different projection directions and are associated respectively with a right eye and a left eye of the person, the method comprising:
providing a visual capacity data of the right eye, of the left eye, or of the right eye and the left eye of the predetermined person;
processing the at least one stereo image such that at least one corresponding image feature in the first partial image and/or the second partial image are each amended in the first partial image and/or the second partial image depending upon the visual capacity data of a respective associated eye of the predetermined person; and
displaying the processed at least one stereo image by a stereoscopic display apparatus.

2. The method of claim 1, wherein a large number of corresponding image features in the first partial image and the second partial image are amended in the first partial image and in the second partial image, respectively, dependent upon the visual capacity data of an associated eye of the predetermined person.

3. The method of claim 1, wherein at least one of the following amendments is carried out: enhancing, attenuating, sharpening, blurring, using high pass, using low pass, filtering, increasing or decreasing depth of focus, increasing or decreasing resolution, transforming, sharpening edges, enhancing contrast, adapting brightness, or removing small details.

4. The method of claim 1, wherein the visual capacity data comprises a contrast resolution capacity, a stereopsis capacity, a resolving power, an accommodation, a color vision capability, an eye motor function, a visual acuity and/or a low-light vision.

5. The method of claim 1, further comprising:
asymmetrical processing of the first partial image and/or the second partial image depending on different visual capacities of the right eye and the left eye.

6. The method of claim 1, wherein a current observation geometry and/or a spacing of the eyes of the person from the display apparatus is captured and taken into account for processing of the at least one stereo image.

7. The method of claim 6, wherein the current observation geometry comprises a size of the display apparatus and/or a viewing angle of the person to the display apparatus.

8. The method of claim 1, wherein data regarding lighting conditions in a region of the person or the display apparatus, and/or optical parameters of the display apparatus are taken into account during the processing of the stereo image.

9. The method of claim 8, wherein the optical parameters comprise visibility, contrast, and/or brightness.

10. The method of claim 1, wherein processing comprises:
forward convolution of the at least one image feature or of an image portion containing the image feature of the first partial image and/or of the second partial image with a function describing the visual capacity of the respective associated eye, so that a perception representation of the image feature by the person results;
amending the perception representation of the image feature through to a desired target perception representation; and
backwards convolution of the desired target perception representation of the amended image feature.

11. The method of claim 1, wherein determining the image features comprises:
analyzing the partial images with regard to corresponding image features and therefrom determining stereo disparities;
creating a depth map of the corresponding image features or the whole scene;
evaluating the detected corresponding image features with regard to at least one evaluation criterion; and
selecting at least one corresponding image feature from a set of the detected corresponding image features on a basis of the at least one evaluation criterion.

12. The method of claim 11, wherein the at least one evaluation criterion has an evaluation regarding a visibility for an average person or a selected person and the corresponding image features which undershoot a threshold value with regard to a visibility criterion are selected.

13. The method of claim 11, wherein a further evaluation criterion is applied which has an evaluation regarding a relevance and the corresponding image features that exceed a threshold value with regard to a visibility criterion.

14. The method of claim 1, wherein to provide the visual capacity data of the person, a vision test is carried out.

15. A system for stereo image processing and representation of at least one stereo image for a predetermined person, the at least one stereo image comprising one first partial image and one second partial image, wherein the one first partial image and one second partial image represent different projection directions and are associated respectively with a right eye and a left eye of the person, the system comprising:
a control unit configured to provide a visual capacity data of the right eye, of the left eye, or of the right eye and the left eye of the predetermined person;
a data processing unit configured to process the at least one stereo image such that at least one corresponding image feature in the first partial image and/or the second partial image are each amended in the first partial image and/or the second partial image depending upon the visual capacity data of the respective associated eye of the predetermined person; and
a stereoscopic display apparatus configured to display the processed at least one stereo image by a stereoscopic display apparatus.

16. The system of claim 15, further comprising:
a test apparatus configured for testing the visual capacity of the eyes of the person.

17. The system of claim 15, further comprising:
at least one sensor configured for measuring an observation geometry; and/or
at least one sensor configured for measuring lighting conditions.

18. The system of claim 15, further comprising:
a medical imaging system configured for recording and providing a medical stereo image.
